(19) United States
(12) Patent Application Publication
(10) Pub. No.: US 2009/0126722 A1
(43) Pub. Date: May 21, 2009

(54) LIQUID EJECTION DEVICE AND EJECTION METHOD

(75) Inventors: Masaru Sugita, Tokyo (JP); Hiroyuki Wada, Machida-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 12/065,693

(22) PCT Filed: Oct. 11, 2006
(86) PCT No.: PCT/JP2006/320686

§ 371 Date: Mar. 4, 2008

(30) Foreign Application Priority Data
Oct. 18, 2005 (JP) 2005-302629

(51) Int. Cl. A61M 11/00

(52) U.S. Cl. 128/200.19

(57) ABSTRACT

A liquid ejection device, includes: a plurality of storing parts for storing liquids; a plurality of liquid-feeding parts connected to the respective storing parts; a ejection part for ejecting a liquid; a plurality of flow paths connected to the plurality of liquid-feeding parts, respectively; and a connection part for joining the plurality of flow paths and connecting them as a single flow path to the ejection part. The plurality of liquid-feeding parts are actuated to mix the liquids from the plurality of storing parts in the single flow path joined at the connection part and feed a mixture of the liquids to the ejection part to eject the mixture from the ejection part. According to the ejection device of the present invention, the ejection amount or the mixing ratio can be easily changed in a comparatively precise manner.

### TECHNICAL FIELD

The present invention relates to an ejection device and an ejection method for ejecting a liquid. More specifically, the present invention is applicable to an ejection mechanism to be utilized in an equipment for generating predetermined airflow under atmospheric pressure, including liquid-atomizing medical equipment such as inhalers and feeders for nasal drops, or air conditioners, air cleaners, ventilating facilities, and air-inlet facilities. As described above, the present invention relates to a liquid ejection device and a liquid ejection method, which can be extensively applicable depending on usage forms thereof.

### BACKGROUND ART

Various kinds of equipments for generating a predetermined air flow under atmospheric pressure have been known in the art, extending from industrially applicable equipment to house-hold equipment. In addition, variations include a wide variety of equipment such as those for air supply, air suction, air replacement, air purification, or supply of a certain component in the flow of air. For instance, an air cleaner is a typical one that is used to purify air in the room and to successively replace the indoor air with flesh outdoor air. In addition, an inhaler, which can be used for supplying various kinds of pharmaceutically effective components in a mist form to an affected part of the respiratory organ, is an example of equipment for entraining a certain component in the flow of air.

Atomizing mechanisms for generating minute liquid droplets in a mist from a liquid include a spray system such as one described below. Like an atomizer for perfume, this system utilizes a pressure difference to be generated when pressurized air passes through a narrow flow path to draw up a liquid through a capillary tube and then atomizes it in mist to eject. Such the spray type atomizing mechanism employs pressurized air as a driving force for sucking and atomizing the liquid passing through the capillary tube. A method of generating pressurized air may be selected from various methods depending on its usage, such as one using a hand pump and one using an electric compressor.

In addition, there is also an ultrasonic mist-generating mechanism. This mechanism ultrasonically generates air bubbles in a liquid and then utilizes a repulsive force, which can be generated when fine air bubbles are collapsed on the surface of the liquid to eject minute liquid droplets. Such the mechanism has been utilized in an ultrasonic humidifier, or the like.

Further, as a mechanism for generating minute liquid droplets, there are other systems such as the inkjet system those major methods are vibration and thermal inkjet system. In the vibration system, for example, a system that utilizes a piezoelectric element or the like has been known in the art. On the other hand, in the thermal inkjet system, for example, a system that utilizes a micro-heater element has been known in the art. In this case, a small amount of pressure is electrically generated and applied on a liquid stored in a liquid receiver to eject fine droplets from a narrow ejection orifice. The mechanisms for generating minute liquid droplets in the vibration and thermal inkjet systems, which can be electrically controlled, have an advantage in that an extremely small amount of liquid droplets can be ejected with high precision by controlling a small amount of pressure to be generated. Using this advantage, applications of such mechanisms have been facilitated in some fields that utilize an extremely small amount of liquid droplets.

For instance, realization of treatment on a user, which makes use of a device for ejecting a medicine to allow the user to take the medicine by inhalation in combination with information database such as electronic medical records, is in progress. An application of the inkjet system to an ejection mechanism in a medicine-ejection device makes it possible to precisely control the diameter of liquid droplets or an ejection amount of a medicine solution (see International Publication Nos. WO 95/01137 and WO 02/04043).

Besides, many of ejection and atomizing devices are provided for ejecting or atomizing a plurality of substances, rather than ejecting or atomizing a single composition. Even in the case of the above medicine-ejection device, various combinations of medicines and auxiliary substances or constructions for ejecting or atomizing a plurality of medicines can be considered. Among such various conceivable combinations, some of them may be difficult to retain respective substances in a mixture, though they are expected to be ejected or atomized in a mixed state. In other words, each substance may lose its original beneficial effects as a result of reaction with others in mixture. Such a case may correspond to a combination having a short pot life. Here the term “pot life” refers to a time period in which a composition provided as a mixture of a plurality of substances is available for intended use. For ejecting or atomizing the combination of substances as described above, a system in which substances are independently stored in their respective containers and then mixed just before ejecting or atomizing the combination of substances has been proposed (see International Publication No. WO 2004/007346). The system, by which solutions are mixed just before being ejected or atomized, allows a combination having a short pot life to be ejected in mixture. However, the system described in International Publication No. WO 2004/007346 has not been clarified with respect to the process from mixing to atomizing or ejecting, so it is hardly constructed to precisely control an amount of ejection. In addition, for controlling a mixture ratio, there is a need of changing the inner pressure of each container, the opening diameter of a liquid-supplying port, or the like. Therefore, the above system is not designed for suitably, easily making a change in a mixture ratio as needed with precise control.

### DISCLOSURE OF THE INVENTION

In view of the above-mentioned problems, an object of the present invention is to provide a liquid ejection device, with which an ejection amount or a mixture ratio can be easily changed and controlled in a comparatively precise manner, when substances having short pot lives are mixed and ejected in mixture.

A liquid ejection device according to the present invention includes: a plurality of storing parts for storing liquids; a plurality of liquid-feeding parts connected to the respective storing parts; an ejection part for ejecting a liquid; a plurality of flow paths connected to the plurality of liquid-feeding parts, respectively; and a connection part for joining the plurality of flow paths and connecting them as a single flow path to the ejection part. In the liquid ejection device, the plurality of liquid-feeding parts are actuated to mix the liquids from the plurality of storing parts in the single flow path joined at the connection part and feed a mixture of the liquids to the ejection part to eject the mixture from the ejection part.

Further, an ejection method according to the present invention actuates a plurality of liquid-feeding parts to allow liquids to respectively pass through flow paths from a plurality of storing parts for storing the liquids, and to join together in a single flow path formed by joining the flow paths. Then, the plurality of liquids is mixed, and the mixture is fed to a ejection part to eject the mixture from the ejection part.

According to the present invention, the plurality of liquid-feeding parts are actuated to allow the liquids stored in the respective storing parts to be mixed together in a single flow path formed by joining their respective flow paths. Therefore, even if the combination is of a short pot life, it can be mixed and ejected. In addition, for example, the control of the drive frequency of the liquid-feeding part or the like enables to easily change the ejection amount and control the mixture ratio thereof in a comparatively precise manner.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a diagram showing an embodiment of a general configuration of a ejection device according to the present invention.

FIG. 2 is a diagram showing an example of a joined part of flow paths of the ejection device according to the present invention.

FIG. 3 is a diagram showing an embodiment of a general configuration of the ejection device according to the present invention.

FIG. 4 is a cross-sectional diagram showing an example of an ejection part of the ejection device according to the present invention.

FIG. 5 is a perspective diagram showing an appearance of the ejection device according to the present invention.

### BEST MODES FOR CARRYING OUT THE INVENTION

Preferred embodiments of the present invention will now be described in detail in accordance with the accompanying drawings.

Hereinafter, embodiments of a ejection device and an ejection method according to the present invention will be described.

According to the embodiments of the present invention, a plurality of liquid-feeding parts are actuated to flow liquids from the respective storing parts through flow paths into a single flow path formed by joining the individual flow paths together. In the single flow path, the plurality of liquids are mixed and the resulting mixture are then ejected. Any of ejection methods may be employed, or any of spray systems or the like, which have been extensively used for liquids of aqueous solvents, may be employed. Preferably, however, any of systems that ejection liquids by application of electric energies may be employed, in particular, a method of ejecting a mixture liquid on the basis of a principle of a thermal inkjet system. In this embodiment, which employs an ejection system of this type, there is employed a mode in which a step of converting a liquid into fine liquid droplets and a step of mixing the fine liquid droplets ejected in an air flow for feeding the liquid droplets are separately performed.

In atomization using the spray system, a pressurized gas used in the process of converting a liquid into the fine liquid droplets is also used for a subsequent air flow for feeding the ejected fine liquid droplets. Therefore, it is structurally difficult to change the amount (i.e., density) of the fine liquid droplets to be floated in the supply air flow or the mixture ratio of compositions depending on the purpose. In contrast, an ejection method of this embodiment employs the above separation mode, so a theoretical limitation to the atomization using the spray system can be avoided. Therefore, it allows a precise controlling of ejection amount or mixture ratio, which is a characteristic feature of the present invention.

Basically, for example, the ejection method of this embodiment may be a method of atomizing a liquid that contains a plurality of pharmaceutical compounds in an aqueous solvent at predetermined concentrations and at predetermined proportions. The liquid may contain an ejection stabilizing agent, at least one of a scenting component and a flavoring component, in the like in homogenous distribution in the aqueous solvent in addition to the pharmaceutical compounds.

By employing the above atomizing method, this embodiment may be also constructed as an atomizer available for atomization of a liquid containing predetermined concentrations of the plurality of pharmaceutical compounds in an aqueous solvent. The atomizer has parts (storing parts) for storing liquids, parts (liquid-feeding parts) for feeding minute amounts of liquids, a flow path for mixing the liquids from the plurality of storing parts, and an ejection part for ejecting fine liquid droplets of the liquids by means of a thermal inkjet system.

An ejection and atomization method and an atomization device of this embodiment are suitable when they applied to an inhaler used for medical care purposes. The inhaler of this embodiment sprays a liquid that contains the pharmaceutical compound, which can be used for a therapeutic purpose, in an aqueous solvent at a predetermined concentration to allow a target person to be administered to inhale the atomized liquid. In this case, the above atomization device is used as an atomization mechanism for carrying out atomization of the liquid. Further, the atomization device is configured such that the above atomization mechanism is provided with an inhalation mechanism for allowing the target person to be administered to inhale a gas in which fine liquid droplets of the liquid generated by the atomization is floated in mist.

The inhaler of this embodiment is configured such that even medicines having short pot lives can be stored in separation and then mixed together just before being ejected. For the medicines, too, the inhalation amount and the mixture ratio of the medicines to be inhaled should be under precise control, so this embodiment can be also suitable in this aspect. In addition, the atomization method of this embodiment can confirm mixing of a liquid in atomization and the subsequent entrain air flow by means of a sense of smell or taste by containing a scenting component or a flavoring component in one storing part. The materials for the storing parts, flow paths, and liquid-feeding parts may be any of materials, and suitably selected from glass, plastic, and metal.

The liquid to be used in this embodiment may be any of those having fluidity and property of allowing the liquid-feeding part to feed the liquid. A main medium is preferably water or an organic material, and, in case of administration to the living body, more preferably, the main medium is water.

Components of the liquid may be any of those that can be homogenous in the liquid, and the state of being homogenous in the liquid may be any of dissolution, dispersion, emulsification, suspension, slurry, and so on.

The liquid composition may be any of those having the above states without distinction of an organic or inorganic matter. As for the compound, any known medicines, flavors, pigments, and so on can be used. The medicines may include any of those for pharmaceutical compounds having pharmacological and physiological actions, components for scenting- and flavoring-purposes, dyes, pigments, and the like.

For the pharmaceutical compounds having the physiological actions, medicine compounds generally used in the art include anti-inflammation steroids, sedatives, β-sympathetic agents, anti-histamine agents, anti-allergic agents, vitamin agents, hypertensive agents, anti-anxiety agent, anti-rheumatic agents, protein preparations, hormonal agents, receptors, antibodies, enzymes, vaccines, genes, and nucleic acids.

The appropriate dose (concentration) of the medicine depends on the kinds of components thereof and may be preferably selected in the range of 1 ppm to 10% based on total amounts of blended ingredients, more preferably selected in the range of 0.001% to 5%. For the above flavoring or scenting component, any of various kinds of natural flavors, synthetic flavors, and preparative flavors can be used. In addition, any of typical flavor components, which have been used in cosmetic flavors, soap flavors, food flavors, and so on, can be used. Examples of secondary components, which may be added, include those for pharmaceutical applications as described in the pharmacopeia of each country or those permitted to be used in foods, cosmetics, and so on.

In general, the blending ratio of a flavor or the like to be mixed as the above flavoring or scenting component is preferably in the range of 1 ppm to 10% based on total amounts of blended ingredients, more preferably in the range of 1 ppm to 1%, which varies depending on the kind of the flavor or the like to be mixed as the above flavoring or scenting component used. In addition, as far as within the range that works for intended purpose of an ejection liquid, both the taste- and scenting components may be used in combination.

For the above dye or pigment, any of various kinds of dyes and pigments can be used. Minor components, which may be added, include those for pharmaceutical applications as described in the pharmacopoeia of each country or those permitted to be used in foods, cosmetics, and so on.

In general, the blending ratio (concentration) of a coloring agent to be mixed as the above dye or pigment is preferably in the range of form 1 ppm to 30% based on total amounts of blended ingredients, more preferably in the range of from 0.01% to 10%, which varies depending on the kind of the coloring agent to be used. In addition, both the dye and the pigment may be used in combination as far as the blending percentage falls within the range that works for intended purpose of an ejection liquid.

Additionally, an additive such as an ejection-assisting agent or an absorption-enhancing agent may be used if necessary. The above medicine, aromatic substance, or coloring agent is a hydrophobic substance that does not represent a desired solubility. In that case, a dispersant, a surfactant, or the like, which can be used for attaining a uniform distribution of components, may be added if necessary. Further, if required, appropriate amounts of various additives that comply with the intended use of the atomization liquid such as a dispersant, a surfactant, a surface tension controlling agent, a viscosity controlling agent, a solvent, a humectant, and a pH controlling agent may be used.

Specific examples of the additives that may be blended include an ionic surfactant, a nonionic surfactant, an emulsifier, a dispersant, a hydrophilic binder, a hydrophobic binder, a hydrophilic thickener, a hydrophobic thickener, glycerin, glycols, glycol derivatives, alcohols, an amino acid, urea, an electrolyte, and a buffer component. Note that one of the various kinds of the additives may be added alone or a plurality of kinds of additives may be added as required.

Various substances to be used as the additives as exemplified above are more preferably those used for pharmaceutical applications as described in pharmacopoeia of each country as secondary components that can be added or those allowed to be used in foods or cosmetics.

The addition ratio (mass concentration) of each substance to be blended as the additive differs depending on the medicine compound as the intended main component, and the type of a flavor used as a flavoring component or a scenting component, the type of a coloring agent, and the mixture ratio thereof. Preferably, the addition ratio is set in the range of from 0.01% to 40% based on total amounts of blended ingredients, and more preferably, in the range of from 0.1% to 20%.

Meanwhile, an addition amount of the additive varies depending on the use (function), type, and combination of the additives, but is preferably selected in the range of 0.5 parts by mass to 100 parts by mass with respect to a total amount of the medicine, and the scenting component or the flavoring component, and the coloring agent of the liquid as 1 part by mass from the viewpoint of ejection property of the liquid to be blended.

The liquid compositions to be filled in the plurality of storing parts are ones selected from those described above and the individual liquids may be made of the same or different substances, or may be a combination of different substances. Specifically, it may be a combination of medicines or a combination of a medicine with a surfactant. In addition, the individual medicine compositions for the plurality of storing parts may be mixtures of medicines, flavors, or pigments with additives, or mixtures of substances selected from medicines, flavors, and coloring agents.

By making a liquid composition that fills the respective storing parts as a combination of different kinds of medicines, a plurality of objects can be attained by one operation. Specifically, it can be exemplified by a case in which a therapeutic agent for respiratory illness and a systemic administration agent for internal treatment are joined together.

Alternatively, a combination of the therapeutic agent with an inhibitor of an enzyme that decomposes the therapeutic agent may be effective. In addition, a combination of a respiratory inhibitor with a systemic administration agent may be also effective. Further, a combination of the therapeutic agent with an absorption-enhancing agent attains the more effective absorption property. In addition, the atomization can be confirmed by joining the therapeutic agent with a flavor.

A combination of a plurality of flavors and medicines for aromatherapy can bring about new effects as well as a more diverse range of effects. In addition, a combination of a medicine with an ejection stabilizing agent can generate their mutual action for the first time just before ejection. Consequently, the liquid can be stored alone until just before ejection, so the stability thereof can be secured.

The liquid-feeding part may be any of mechanisms that can feed the liquid, but a mechanism capable of feeding a fine amount of a liquid, such as a micropump is preferably used. Examples of a system for producing driving force to generate the liquid feeding include a pressure extrusion system, a thermal inkjet system, and a vibration system. In addition, the control of such the system may be of manual or electronic. In the case of electric control, it is more preferable that it can be optionally controlled by a program that controls the amount of the liquid more precisely. Examples of an electric control system include vibration systems such as a piezoactuator system and an ultrasonic system, and the thermal inkjet system realized through application of thermal energy.

For the liquid-feeding part, it is more preferable to provide an inlet that is connected to the storing part and an outlet that is connected to the confluence of flow paths with check valves, respectively, for controlling the amount of the liquid precisely while preventing the reverse flow thereof.

The ejection device of this embodiment has an ejection part based on the principle of a thermal inkjet that permits the ejection of a fine droplet of a liquid by a thermal bubbling system. In this case, it is preferable that multiple parts for ejecting liquids, which constitute the heard part, are designed to actuate independently.

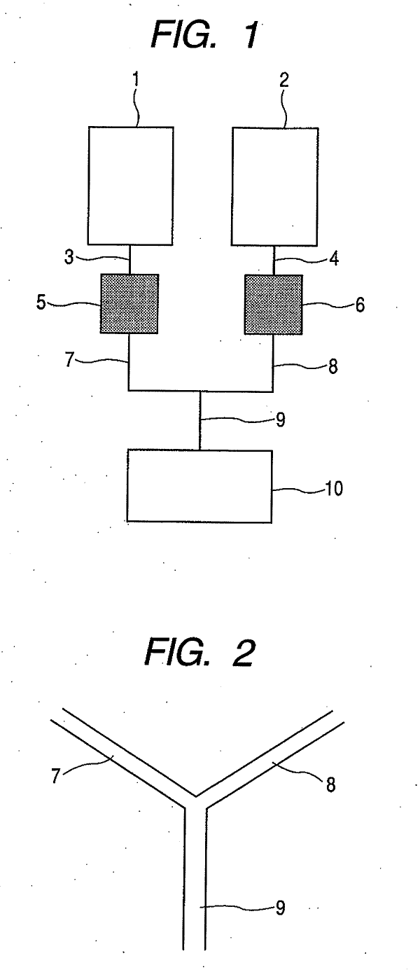

FIG. 1 schematically illustrates an example of a general configuration of such the device for liquid ejection and atomization. The device exemplified in FIG. 1 includes a plurality of storing parts 1 and 2 for storing liquids, a plurality of liquid-feeding parts 5 and 6, a flow path 3 for connecting the storing part 1 and the liquid-feeding part 5, and a flow path 4 for connecting the storing part 2 and the liquid-feeding part 6. Further, the device further includes a ejection part 10 and flow paths 7 to 9 that connect the ejection part 10 with the liquid-feeding parts 5 and 6. The flow path 9 is one formed by joining the flow path 7 with the flow path 8. In this configuration, the liquid-feeding parts 3 and 4 are actuated to allow liquids to be mixed together at the connection, that is, the flow path 9, and a mixture liquid is then fed to the ejection part 10, followed by ejecting the mixture liquid from the ejection part 10. On this occasion, for preventing the reverse flow, it is preferable to arrange the flow paths 7, 8, and 9 in the form represented in FIG. 2. The flow paths 7, 8, and 9 have sufficiently narrow diameters, so the effects of preventing the reverse flow will not vary depending on the posture of the device in use.

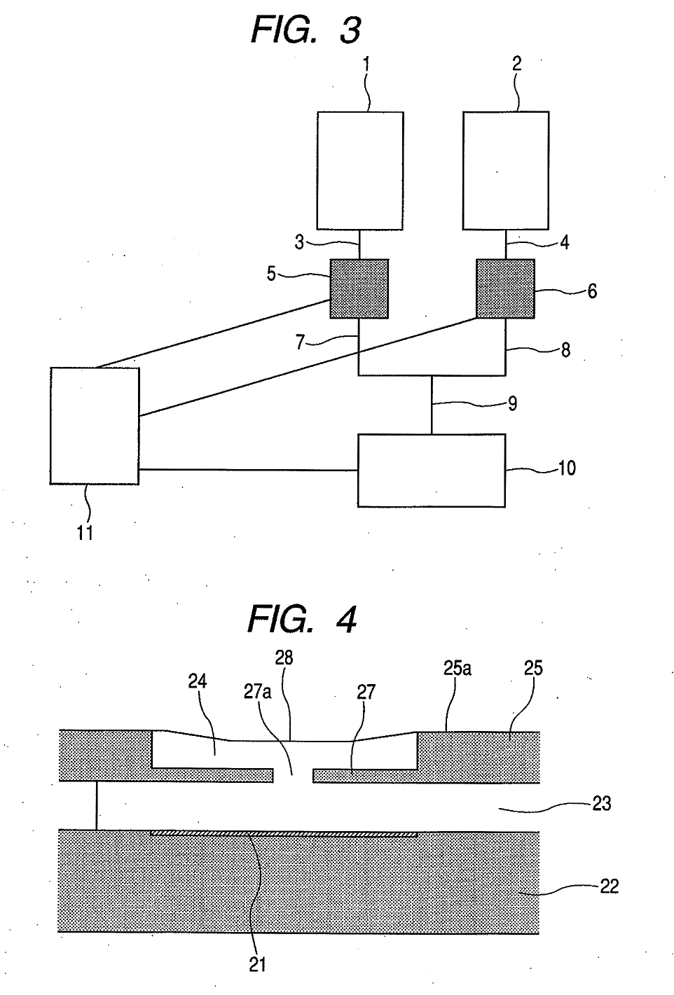

The ejection part 10 has a ejection part. As shown in FIG. 3, the ejection part connects with a controller 11 for controlling the driving of a liquid ejection part through inner wiring to exchange driving signals, control signals, and so on. Note that, the same reference numerals as those shown in FIG. 1 depict identical members as shown in FIG. 1, respectively.

In addition, the controller 11 further connects with the liquid-feeding parts 5 and 6 to control the actuation of the respective liquid-feeding parts. The actuation controls include the control of an actuation period, the control of a drive frequency, and the control of actuation timing. By making the variations in actuation frequencies or drive-time period of the respective liquid-feeding parts to make a change in mixture ratio easily, or to carry out the control of drive frequency or drive-time period, so the control of blending ratio can be also controlled comparatively in a precise manner.

The method of actuating each liquid-feeding part is not specifically limited as far as it is able to mix the liquids from the respective storing parts at the flow path 9. In other words, any of actuation methods can be allowable, except that the mixing of the liquids does not occur in the case where a necessary ejection amount of the liquid is fed from one storing part after a necessary ejection amount of the liquid is completely fed from another storing part.

However, for more uniformly mixing the liquids, it is preferable to actuate each of the liquid-feeding part such that the desired ejection amounts of the respective storing parts are divided, and then the liquid can be alternately supplied to the flow paths 9. For example, it corresponds to the case in which each liquid-feeding part is individually actuated and the switching of the actuation is alternately carried out at millisecond orders. In this case, the shorter the intervals of switching becomes, the more uniform mixture becomes. Alternatively, also in the case of carrying out the liquid feeding at the same period of time without switching the actuation of the respective liquid-feeding parts, as long as the actuation of the respective liquid-feeding parts are controlled so that their actuation timings are shifted, the supply of a liquid from each flow path to the flow path 9, can be alternately carried out. In particular, in the case where the drive frequencies of the respective liquid feeding parts are the same, and the actuations thereof are carried out at the same time zone, it is preferable to control so that the actuate timing of the respective liquid feeding parts may shift. In this case, if the actuate timings also coincide to each other, there is a fear in that the mixing cannot be carried out sufficiently and uniformly in the flow path 9.

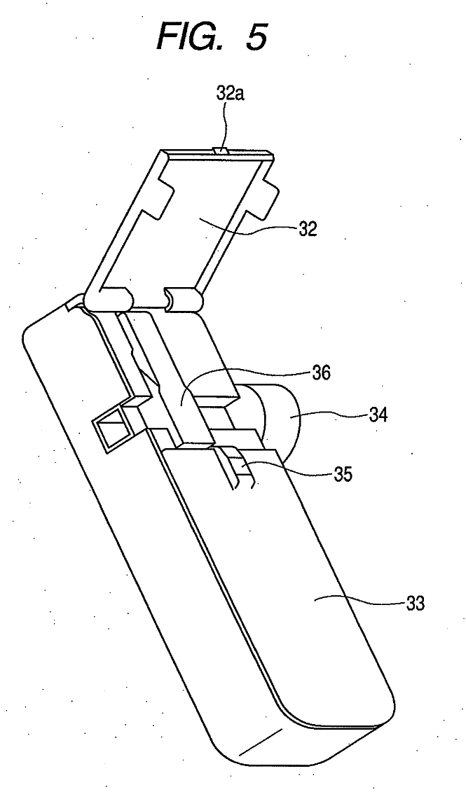

The ejection part may preferably utilize a part for ejecting minute liquid droplets, which has excellent controllability in addition to the ability of providing each of fine liquid droplets ejected thereof with a liquid amount in the order of subpicoliters or femtoliters. The ejection head may be one as disclosed in JP 2003-154655 A. The ejection head described above will now be described with reference to the cross sectional diagram of FIG. 4.

As shown in FIG. 4, an orifice 24 is provided with a contraction part 27 at a location where a recess is formed with respect to an orifice surface 25a, the surface of an opening of the orifice 24 formed in a orifice plate 25. In other words, the contraction part 27 is formed by remarkably narrowing the cross sectional area of one part of the orifice 24, compared with other parts thereof. A liquid to be ejected is retained in the orifice 24 by forming a meniscus 28 at an interface between the orifice surface 25a and the contraction part 27. Therefore, the contraction part 27 is located in a liquid in a passage from a liquid flow path 23 to the orifice surface 25a of the orifice plate 25. On a substrate 22, there is provided a heater 21 that generates heat when a voltage is applied thereon for ejecting the liquid. When the heater 21 generates heat, an air bubble is formed thereon to allow liquid droplets to be ejected from the meniscus 28.

In FIG. 1, there is shown an example having two kinds of different liquids to be ejected. If there are three or more different liquids to be ejected, it is possible to cope with such the case by appropriately providing the corresponding storing parts and connecting them with each other, while configuring the head of the ejection part 10 to accumulate ejection parts for several kinds of different liquids.

The ejection device of this embodiment may have an outer shape as shown in FIG. 5. This is configured so that it allows the ejection device to be used as an inhaler, while being designed to be portable for allowing a user to carry. FIG. 5 represents an open state of an access cover 32 of the ejection device to be used as an inhaler. In FIG. 5, the reference numeral 33 is a front cover, which forms a housing together with the body of the ejection device and 35 denotes a lock lever. This is formed such that a protruded part 32a provided on the tip of the access cover 32 hooks on a craw-shaped part formed on the tip 5 of the lock lever 35 and spring-biased to prevent the access cover 32 to be opened at the time of use. When the lock lever 35 is slid downward, the access cover 32 is opened relative to a hinge shaft as its rotation center by the force of a return spring biasing the access cover 32 (not shown).

As shown in FIG. 5, when an access cover 32 is opened, an ejection unit 36 and a mouthpiece 34, which are mounted in a housing become exposed along a ejection unit guide. The mouthpiece 34 is mounted below the ejection unit 36, and are intersected with each other. The ejection unit 36 is constructed of a storing part containing liquids, liquid-feeding parts, various flow paths as described above, a ejection part for ejecting a mixture liquid, an electrical connection plate for supplying electric power from a battery to a heater formed on the ejection part to cause thermal energies from the heater provided on the ejection part, and so on.

For utilizing the ejection device of this embodiment as a device for screening compounds or the like, the device configured as described above can be also suitably employed.

According to this embodiment, for convenience, the device can be used multiple times and certain parts thereof can be replaced with new parts for every use as a cartridge. As for the part, which can be provided as a cartridge, for example, the whole parts shown in FIG. 1 may be provided as a single cartridge, or parts 1 and 2 that contain the liquids may be provided as independent cartridges, or the ejection part 10 may be provided as a cartridge. Further, in FIG. 1, the device may be configured such that the upper part from the ejection part 10 may be regarded as a single member and then provided as a cartridge, or each of the lower parts from the parts 1 and 2 that contain liquids may be regarded as a single member and then provided as a cartridge.

The ejection device of this embodiment uses the advantage of the ejection method of this embodiment. In other words, it takes advantage of the mode in which the step of converting a liquid into fine liquid droplets is separated from the step of mixing fine liquid droplets ejected into the air flow for feeding such liquid droplets. As described above, in this case, a liquid containing an aqueous solvent at a predetermined concentration of a medicine compound used for therapeutic purposes is ejected and a subject to be administered then inhales the liquid ejected, an amount (dose per single administration) of the medicine compound in a gas to be inhaled can be arbitrarily set. In this case, an ejection head based on a thermal inkjet principle having orifices of fine droplets arranged at high density per unit area can be used as an ejection mechanism for ejecting the liquid, thereby allowing size reduction of the atomizer for portable use.

When the liquid for ejection as described above is used for inhalation into the lungs, an indispensable part is a ejection part which can eject a formulation in liquid droplets having particle diameters of 1 to 5 μm with a narrow particle size distribution. In this configuration, as described above, the ejection part, the storing parts, the liquid-feeding part, the connection part, and the flow paths can be removably arranged in appropriate units. The ejection device as shown in FIG. 5, which is designed to be portable for allowing a user to carry the ejection device, is an example of an inhaler capable of ejecting a liquid as liquid droplets having an almost uniform particle size at a constant amount.

In addition, the use of the device of this embodiment makes it possible to sense the reaction between different kinds of substances and a mutual action thereof. For example, a solution containing a substance to be detected can be ejected in the same pattern on a substrate to allow the substrate and the substance to be detected to effectively react with each other or to make variations in their concentrations by only changing the ejection amounts.

### EXAMPLES

Hereinafter, examples of the present invention will be described. However, the present invention is not limited to these examples. The case, in which a mechanism shown in FIG. 3 and an inhaler shown in FIG. 5 were employed and two storing parts were provided for storing liquids, was carried out. The contents of the examples are listed in Table 1 and the contents of the comparative examples are listed in Table 2.

### Example 1

Storing parts 1 and 2 were filled with a solution A and a solution B described below, respectively. In addition, piezoelectric pumps were used in liquid-feeding parts 5 and 6, respectively. The pump is specified that 5 μl of liquid can be fed when the pump is actuated at 50 Hz for one second. For the actual actuation of the pumps, the frequencies of the pumps for solutions A and B were respectively set to 40 and 10 Hz and simultaneously actuated for one second to feed 4 μl of the solution A and 1 μl of the solution B, respectively.

Solution A: an aqueous solution of 4 mg/ml insulin

Solution B: an aqueous solution of 20 mg/ml lauroyl sarcosine

A ejection part 10 having 400 nozzles of 3 μm in nozzle diameter was used. The mixture liquid was atomized for one second and then re-atomized with 4 seconds intervals, thereby carrying out atomization 50 times in total. For repeating atomization, the liquid feeding and liquid supply were appropriately carried out. Further, the characteristics of the device at the time of atomization under the above conditions were confirmed.

The state of atomization was visually observed and, as a result of determination of the number of atomization realized among 50 times of atomization, 50 times of atomization were realized.

In addition, as a result of measuring the particle size distribution of the atomized liquid using a particle size distribution analyzer (manufactured by Malvern, Co., Ltd., Spray Stick), the particles had a mean particle size of 3.3 μm.

In addition, the atomized liquid was collected and a calibration curve of concentration was prepared in advance using a high-performance liquid chromatography (manufactured by JASCO Corporation), followed by determining the concentrations of two substances, the above solutions A and B. As a result, the ratio of the ejection liquid, which had been charged and fed, was 0.8.

In all of the following examples, to uniform the drive-time period of the respective liquid-feeding parts, the amounts of the respective components in the solutions A and B are determined by two factors, concentration and frequency, to define the amount of the substance. In other words, for starting and ending the feed of solutions A and B simultaneously, the amounts of the respective components are adjusted by the frequencies. The ratio of the ejection liquid can be calculated by the following calculation equation.

(Amount of component in A)=(Concentration of component in A)×(drive frequency of liquid-feeding part for A)

(Amount of component in B)=(Concentration of component in B)×(drive frequency of liquid-feeding part for B)

Ratio of ejection liquid=(amount of component in A)/(amount of component in B)

### Comparative Example 1

Comparative Example 1 was carried out in the same way as that of Example 1, except that the feed of solution A was only carried out at first and then the feed of solution B was only carried out. As a result, the atomization was completed 15 times.

### Comparative Example 2

Comparative Example 2 was carried out in the same way as that of Example 1, except that the feed of solution B was only carried out at first and then the feed of solution A was only carried out. As a result, the atomization was completed 20 times.

The solution B is an additive that contributes to stabilize the ejection of the solution A (insulin solution). Therefore, in Example 1, it was found that both solutions are uniformly mixed.

### Example 2

In Example 1, the substance in the solution B was changed to arginine, the set value of the pump was changed to 40 Hz, and the amount of a liquid to be fed was changed to 4 μl. As the both liquid-feeding parts had the same drive frequency, the liquid-feeding parts were controlled so that their actuation timings were staggered. In addition, the same evaluation by atomizing as that of Example 1 was carried out after two-month storage of the solutions A and B at 30° C. As a result, all of 50 atomization trials were succeeded. In addition, the ratio of 0.08 was obtained with respect to the concentration ratio of the liquid, which was mixed and fed.

### Examples 3 to 5

In Example 2, the contents of the liquid was changed to those represented in Table 1 and then evaluated in the same way as that of Example 2. The results obtained are shown in Table 1. That is, it was found that the mixture ratio could be controlled precisely.

### Comparative Example 3

In Example 1, the compositions and the mixture ratios of the solutions A and B were identical and then mixed and stirred in a vial. Subsequently, the mixture solution was stored for two months at 30° C. and then placed in the solution-A-containing part (storing part) 1 of Example 1 to feed the liquid to the ejection part 10, thereby ejecting the liquid from the ejection part. As a result, no ejection was observed.

### Comparative Examples 4 to 7

Evaluation was carried out in the same way as that of Comparative Example 3, except that the contents thereof were changed to those represented in Table 2 below. As a result, no ejection was observed.

From the above, by mixing a plurality of liquids just before ejection, it was found that the device was extremely excellent in storage stability and ejection property.

### Examples 6 to 15

Substances to be contained in the respective liquid storing parts 1 and 2 and the concentrations thereof as well as the proportions of the respective substances contained in an ejection liquid were listed in Table 1, respectively. A medium used was purified water and each of the substances was then prepared at concentrations shown in the table.

In addition, on the basis of known information, the concentrations of the respective substances after ejection were calculated from an absorbance ratio obtained by a spectral measurement using a spectral photometer (manufactured by JASCO Corporation, V-560) or a peak area ratio of chromatogram obtained using high-performance liquid chromatograph. As for the examples in which two components are represented in the solutions A and B in Table 1, the proportions of the respective substances other than the second component provided as an additive are represented as the ratio after the ejection. In any of systems, it was confirmed that the desired substances can be ejected and atomized at any ratio and the amounts thereof can be controlled with high precision, and the plurality of substances can be mixed just before ejection.

The present invention is not limited to the above embodiments and various changes and modifications can be made within the spirit and scope of the present invention. Therefore to apprise the public of the present invention, the following claims are made.

TABLE 1

| | Solution A | | | | | Solution B | | | | | | |
|---|---|---|---|---|---|---|---|---|---|---|---|---|
| | | | | | Amount | | | | | Amount | | |
| | | | | | of | | | | | of | | Ejection |
| | | | | Pump | liquid | | | | Pump | liquid | | Liquid |
| Example | Species | Concentration | | Frequency | fed | Species | Concentration | | Frequency | fed | Atomizability | A/B |
| 1 | Insulin | 4 | mg/ml | 40 | 4 | Lauroyl | 20 | mg/ml | 10 | 1 | 50 | 0.8 |
| | | | | | | sarcosine | | | | | | |
| 2 | Insulin | 4 | mg/ml | 40 | 4 | Arginine | 50 | mg/ml | 40 | 4 | 50 | 0.08 |
| 3 | Insulin | 4 | mg/ml | 40 | 4 | Arginine | 100 | mg/ml | 40 | 4 | 50 | 0.04 |
| 4 | Insulin | 4 | mg/ml | 40 | 4 | Lauramidopropyl | 5 | mg/ml | 40 | 4 | 50 | 0.80 |
| | | | | | | betaine | | | | | | |
| 5 | Insulin | 10 | mg/ml | 40 | 4 | Lauramidopropyl | 50 | mg/ml | 10 | 2 | 50 | 0.40 |
| | | | | | | betaine | | | | | | |
| 6 | Insulin | 4 | mg/ml | 40 | 4 | Lauroyl | 10 | mg/ml | 10 | 1 | 50 | 1.60 |
| | | | | | | sarcosine | | | | | | |
| 7 | Growth | 5 | mg/ml | 50 | 5 | Lauroyl | 50 | mg/ml | 20 | 2 | 50 | 0.25 |
| | hormone | | | | | sarcosine | | | | | | |
| 8 | Insulin | 10 | mg/ml | 40 | 4 | Lauroyl | 50 | mg/ml | 10 | 2 | 50 | 0.41 |
| | | | | | | sarcosine | | | | | | |
| 9 | Albumin | 1 | mg/ml | 20 | 2 | Cocamide | 20 | mg/ml | 20 | 2 | 50 | 0.05 |
| | | | | | | propylbetaine | | | | | | |
| 10 | Insulin/ | 4 | mg/ml/ | 40 | 4 | Salbutamol | 10 | mg/ml | 10 | 1 | 50 | 1.61 |
| | Arginine | | | | | | | | | | | |
| 11 | Insulin/ | 4 | mg/ml/ | 40 | 4 | Menthol | 1 | mg/ml | 20 | 2 | 50 | 7.98 |
| | Arginine | | | | | | | | | | | |
| 12 | Cromoglycic | 5 | mg/ml | 50 | 5 | Menthol | 1 | mg/ml | 20 | 2 | 50 | 12.5 |
| | acid | | | | | | | | | | | |

| | | | | | | | | | | | |
|---|---|---|---|---|---|---|---|---|---|---|---|
| 13 | Insulin | 4 | mg/ml | 40 | 4 | DPP4 Inhibitor/ | 1 mg/ml/ | 40 | 4 | 50 | 2.02 |
| | | | | | | | 50 mg/ml | | | | |
| 14 | GLP-1 | 1 | mg/ml | 20 | 2 | DPP4 Inhibitor/ | 1 mg/ml/ | 20 | 2 | 50 | 1.00 |
| | | | | | | | 50 mg/ml | | | | |

| | | | | | | | | | | |
|---|---|---|---|---|---|---|---|---|---|---|
| 15 | Rosemary | 10 mg/ml/ | 50 | 5 | Citrus | 10 mg/ml/ | 50 | 5 | 50 | 1.01 |
| | extract/ | 50 mg/ml | | | extract/TWEEN80 | 50 mg/ml | | | | |
| | TWEEN80 | | | | | | | | | |

* In Examples 2 to 5, evaluations were carried out after storing each of them at 30° C.

TABLE 2

| | Solution A | | | Solution B | | | | |
|---|---|---|---|---|---|---|---|---|
| Comparative | | | Blending | | | Blending | | |
| Example | Species | Concentration | amount | Species | Concentration | amount | Atomizability | Remarks |
| 1 | Insulin | 4 mg/ml | 4 μl | Lauroyl | 20 mg/ml | 1 μl | 15 | Solutions A |
| | | | | sarcosine | | | | and B were fed |
| | | | | | | | | and atomized |
| | | | | | | | | without |
| | | | | | | | | mixing, in the |
| | | | | | | | | order from A |
| | | | | | | | | to B. |
| 2 | Insulin | 4 mg/ml | 4 μl | Lauroyl | 20 mg/ml | 1 μl | 20 | Solutions A |
| | | | | sarcosine | | | | and B were fed |
| | | | | | | | | and atomized |
| | | | | | | | | without |
| | | | | | | | | mixing, in the |
| | | | | | | | | order from B |
| | | | | | | | | to A. |
| 3 | Insulin | 4 mg/ml | 4 μl | Arginine | 50 mg/ml | 4 μl | 0 | Stored at 30° C. |
| | | | | | | | | after mixing |
| 4 | Insulin | 4 mg/ml | 4 μl | Arginine | 50 mg/ml | 4 μl | 0 | Stored at 4° C. |
| | | | | | | | | after mixing |
| 5 | Insulin | 4 mg/ml | 4 μl | Lauramidopropyl | 5 mg/ml | 4 μl | 0 | Stored at 30° C. |
| | | | | betaine | | | | after mixing |
| 6 | Insulin | 10 mg/ml | 4 μl | Lauramidopropyl | 50 mg/ml | 2 μl | 0 | Stored at 30° C. |
| | | | | betaine | | | | after mixing |
| 7 | Insulin | 4 mg/ml | 4 μl | Arginine | 100 mg/ml | 4 μl | 0 | Stored at 30° C. |
| | | | | | | | | after mixing |

This application claims the benefit of Japanese Patent Application No. 2005-302629, filed Oct. 18, 2005, which is hereby incorporated by reference herein in its entirety.

1-10. (canceled)

11. A medicine ejection device for ejecting a mixture of a plurality of medicines including at least one kind of the medicine for medical treatment, comprising:
a plurality of storing parts for each storing one kind of the medicine;
a plurality of liquid-feeding parts which are connected to said respective storing parts and each of which comprises a micropump;
an ejection part for ejecting the medicines;
a plurality of flow paths connected to said plurality of liquid-feeding parts, respectively; and
a connection part for joining said plurality of flow paths and connecting them as a single flow path to said ejection part,
wherein said plurality of liquid-feeding parts are actuated to mix the medicines from said plurality of storing parts in the single flow path joined at said connection part and feed a mixture of the medicines to said ejection part to eject the mixture from said ejection part.

12. The medicine ejection device according to claim 11 further comprising a controller for alternately and independently actuating said plurality of micropumps.

13. The medicine ejection device according to claim 11, wherein said ejection part ejects the mixture by being applied with an electric energy.

14. The medicine ejection device according to claim 13, wherein the ejection method for ejecting the mixture by applying the electric energy is a method of ejecting the mixture on the basis of a principle of a thermal inkjet system.

15. The medicine ejection device according to claim 11, wherein said ejection part ejects or atomizes the mixture by converting the mixture into liquid droplets.

16. An inhaler for allowing a user to inhale a plurality of medicines as liquid droplets, comprising said medicine ejection device according to claim 11.

17. The inhaler according to claim 16, wherein said inhaler is configured to be portable and capable of being carried.

18. An ejection method, comprising:
actuating a plurality of micropumps to allow a plurality of medicines for medical treatment to respectively pass through flow paths from a plurality of storing parts for each storing one kind of the medicine, and to join together in a single flow path formed by joining the flow paths to obtain a mixture of the medicines; and
feeding the mixture to an ejection part to eject the mixture from the ejection part.

19. The medicine ejection method according to claim 18, wherein said plurality of micropumps are actuated alternately and independently.
